(19) United States
(12) Patent Application Publication
(10) Pub. No.: US 2023/0263942 A1
(43) Pub. Date: Aug. 24, 2023

(54) HIGHLY COMPRESSIBLE SHAPE MEMORY DOUBLE NETWORK HYDROGEL, USE AND PREPARATION METHOD THEREOF, AND INTERVERTEBRAL DISK SCAFFOLD

(71) Applicant: National Tsing Hua University, Hsinchu City (TW)

(72) Inventors: Tzu-Wei Wang, Hsinchu City (TW); Chia-Yu Ho, Hsinchu City (TW)

(73) Assignee: National Tsing Hua University, Hsinchu City (TW)

(21) Appl. No.: 17/707,969

(22) Filed: Mar. 30, 2022

(30) Foreign Application Priority Data
Feb. 23, 2022 (TW) 111106537

(51) Int. Cl. A61L 27/52

(52) U.S. Cl. CPC A61L 27/52; A61L 2400/16

(57) ABSTRACT

A highly compressible shape memory double network hydrogel includes a first network and a second network interpenetrating with each other. The first network is a chemically crosslinked cellulose by chemical crosslinking, and the chemical crosslinking is accomplished by the formation of ether groups between the cellulose. The second network is a physically crosslinked alginate by physically crosslinking, and the physical crosslinking is accomplished by reaction of the alginate with divalent metal ions. In a preparation process of the highly compressible shape memory double network hydrogel, the cellulose and the alginate are mixed first, the chemical crosslinking is then performed to obtain the first network, followed by the physical crosslinking to obtain the second network.

### CROSS-REFERENCE TO RELATED APPLICATION

This application claims the priority benefit of Taiwanese application serial no. 111106537, filed on Feb. 23, 2022. The entirety of the above-mentioned patent application is hereby incorporated by reference herein and made a part of this specification.

### BACKGROUND

### Technical Field

The disclosure relates to a technology of a double network hydrogel, and in particular to a highly compressible shape memory double network hydrogel, use and preparation method thereof, and an intervertebral disk scaffold.

### Description of Related Art

Hydrogel is a material with water as the dispersion medium. A part of hydrophobic groups and hydrophilic residues are introduced into the water-soluble polymer with a network cross-linked structure. The hydrophilic residues combine with the water molecules to link the water molecules inside the network, while the hydrophobic residues expand when exposed to water. Traditionally prepared hydrogels are often composed of a single polymer network structure or a double network structure that is cross-linked by covalent or non-covalent bonds.

The traditional natural polymer hydrogel has low mechanical properties, and if it is used as a biomedical material for hard tissue replacement, it will be easily damaged by extrusion after being implanted into the body wear-bearing site. In addition, some of the current implant surgeries often result in large wound defect, which may influence the recovery rate and cause severe pain.

### SUMMARY

The disclosure provides a highly compressible shape memory double network hydrogel, capable of significantly improving the mechanical strength and compressibility of natural polymer hydrogels.

The disclosure also provides an application of a highly compressible shape memory double network hydrogel for an intervertebral disk scaffold.

The disclosure also provides a method of using a highly compressible shape memory double network hydrogel, capable of being used for the implantation of the intervertebral disk scaffold.

The disclosure further provides a preparation method of a highly compressible shape memory double network hydrogel, capable of producing a double network hydrogel with high compressibility and shape memory effect.

The highly compressible shape memory double network hydrogel of the disclosure includes a first network and a second network interpenetrating with each other. The first network is chemically crosslinked cellulose obtained by chemical crosslinking, and the chemical crosslinking is accomplished by formation of ether groups in the cellulose. The second network is a physically crosslinked alginate obtained by physical crosslinking, and the physical crosslinking is accomplished by reaction of the alginate with divalent metal ions. In a preparation process of the highly compressible shape memory double network hydrogel, the cellulose and the alginate are mixed first, the chemical crosslinking is then performed to obtain the first network, followed by the physical crosslinking to obtain the second network.

In one embodiment of the disclosure, the concentration of the cellulose is 1 to 10 wt. %.

In one embodiment of the disclosure, the double network hydrogel may further include a cross-linking agent for the chemical crosslinking. The concentration of the cross-linking agent is 5 to 10 wt. %.

In one embodiment of the disclosure, the cross-linking agent comprises epichlorohydrin (ECH), poly(ethylene glycol) diglycidyl ether or diglycidyl ether.

In one embodiment of the disclosure, the divalent metal ions are calcium ions, copper ions, ferrous ions, manganese ions, magnesium ions, strontium ions or zinc ions.

In one embodiment of the disclosure, the concentration of the calcium ions (Ca²⁺) is 1 to 10 wt. %, and the concentration of the alginate is 0.5 to 5 wt. %.

In one embodiment of the disclosure, the double network hydrogel may further include a chelating agent.

The intervertebral disk scaffold of the disclosure includes the highly compressible shape memory double network hydrogel.

The method of using the highly compressible shape memory double network hydrogel of the disclosure includes the followings. The highly compressible shape memory double network hydrogel is placed into a mold, and then a chelating agent is added to the highly compressible shape memory double network hydrogel.

The preparation method for a highly compressible shape memory double network hydrogel of the disclosure includes the followings. Cellulose and an alginate are mixed to obtain a mixture. A chemical crosslinking is performed on the cellulose in the mixture to form a hydrogel structure. A physical crosslinking is performed after the chemical crosslinking, so that the alginate in the hydrogel structure reacts with divalent metal ions to form a double network hydrogel.

In another embodiment of the disclosure, a cross-linking agent used in the chemical crosslinking comprises epichlorohydrin (ECH), poly(ethylene glycol) diglycidyl ether or diglycidyl ether.

In another embodiment of the disclosure, the divalent metal ions are calcium ions, copper ions, ferrous ions, manganese ions, magnesium ions, strontium ions or zinc ions.

In another embodiment of the disclosure, before the physical crosslinking is performed, the preparation method further includes shaping the hydrogel structure.

In another embodiment of the disclosure, a chelating agent may also be added to the double network hydrogel after the physical crosslinking, and then the hydrogel structure is shaped, and a solution containing the divalent metal ions is added to the shaped double network hydrogel to achieve a shape-fixed effect.

In another embodiment of the disclosure, the method of mixing the cellulose and the alginate includes adding alginate powder to a cellulose solution.

In another embodiment of the disclosure, before that the cellulose and the alginate are mixed, the preparation method further includes repeatedly freezing and thawing the cellulose solution three to five times.

In all embodiments of the disclosure, the chelating agent includes ethylenediaminetetraacetic acid (EDTA), nitrilotriacetic acid, or hydroxyethylethylenediaminetriacetic acid (HEDTA).

Based on the above, the disclosure uses different cross-linking mechanisms between two natural polymers to achieve the effect of improving the mechanical properties and shape memory effect of materials. Compared with traditional single network hydrogels, the double network hydrogels of the disclosure have stronger mechanical properties and may rebound under high compressive stress, thus having high compressibility. Compared with double network structures that are cross-linked entirely by covalent bonds, the double network hydrogel of the disclosure is suitable for intervertebral disk scaffolds subjected to higher compressive stress because its physical crosslinking part may absorb and dissipate energy by breaking bonds and then re-form new bonds, which may protect the internal structure of the polymer from damage and disintegration of the material.

To make the aforementioned more comprehensible, several accompanied with drawings are described in detail as follows.

### BRIEF DESCRIPTION OF THE DRAWING

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram of a highly compressible shape memory double network hydrogel according to a first embodiment of the disclosure.

FIG. 2A to FIG. 2C are schematic diagrams of a preparation process of a highly compressible shape memory double network hydrogel according to a second embodiment of the disclosure.

FIG. 3 is a stress-strain curve of Experimental Example 1, Comparative Example 1, and Comparative Example 2.

FIG. 4 is a stress-strain curve measured by repeated compression in Experimental Example 1.

FIG. 5 is magnetic resonance imaging (MRI) images of caudal vertebrae of rats of NC, DC, DN, PDN and GPDN.

FIG. 6 is immunostaining (IHC) images of caudal vertebrae of rats of NC, DC, DN, PDN and GPDN.

FIG. 7 is a bar graph of quantification of Vimentin and FOXF1 proteins in the IHC images of FIG. 6.

### DESCRIPTION OF THE EMBODIMENTS

The following provides many different embodiments for implementing different features of the disclosure. However, these embodiments are merely exemplary, and are not intended to limit the scope and application of the disclosure. Furthermore, for the sake of clarity, the relative dimensions (e.g., length, spacing, etc.) and relative positions of the compositions or structures may be reduced or enlarged.

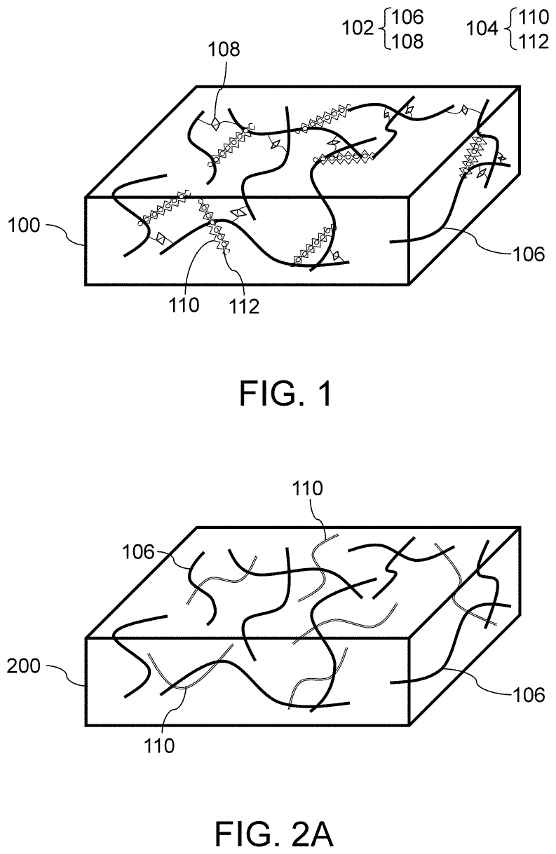

FIG. 1 is a schematic diagram of a highly compressible shape memory double network hydrogel according to a first embodiment of the disclosure.

Referring to FIG. 1, a highly compressible shape memory double network hydrogel 100 according to the first embodiment includes a first network 102 and a second network 104 interlaced with each other. The first network 102 is chemically crosslinked cellulose 106 obtained by chemical crosslinking, and the chemical crosslinking is accomplished by formation of an ester bond 108 in the cellulose 106. A cross-linking agent such as epichlorohydrin (ECH), poly(ethylene glycol) diglycidyl ether or diglycidyl ether may be used. The cross-linking agent with epoxy groups may complete the chemical crosslinking, and the concentration of the cross-linking agent is, for example, in a range of 5 to 10 wt. %. If the concentration of the cross-linking agent is less than 5 wt. %, the proportion of the chemical crosslinking is reduced, which affects the shape memory effect (unable to effectively fix at the permanent shape); on the other hand, if the concentration of the cross-linking agent is greater than 10 wt. %, the cross-linking density is too high, which makes the material unable to be shaped, resulting in a hard and brittle hydrogel material that cannot maintain its original compressible nature. The concentration of the cellulose 106 used in the first embodiment is, for example, 1 to 10 wt. %. If the concentration of the cellulose is less than 1 wt. %, polymer concentration is too low, which may make an aqueous solution difficult to become gel; if the concentration of the cellulose is more than 10 wt. %, high concentration makes the aqueous solution highly viscous and difficult to homogeneously stir the subsequent addition of the cross-linking agent and an alginate solution, resulting in a decrease in the stability of the hydrogel. The second network 104 is a physically crosslinked alginate obtained by physical crosslinking, and the physical crosslinking is accomplished by reaction of an alginate 110 with divalent metal ions 112. In FIG. 1, spheres represent the divalent metal ions 112. The alginate 110 chelates with the divalent metal ions 112 to form the physical crosslinking by conformational coincidence, where the divalent metal ions 112 are, for example, calcium ions (Ca²⁺), copper ions, ferrous ions, manganese ions, magnesium ions, strontium ions zinc ions, etc. The calcium ions are sourced from one of calcium chloride, calcium sulfate, calcium aluminosilicate, calcium carbonate, calcium chloride, calcium oxide, calcium hydroxide, calcium lactate, calcium citrate, calcium gluconate, or a group thereof, and the concentration of the calcium ions is, for example, 1 to 10 wt. %. If the concentration of the calcium ions is greater than 10 wt. %, overall noncovalent bonding concentration of the hydrogel increases, resulting in the overall brittle and hydrophobic nature of the hydrogel, the shape memory properties becomes unclear (the calcium ions cannot be effectively removed to achieve an effect of shape recovery), and excessive concentration of the calcium ions also leads to cytotoxicity. On the other hand, if the concentration of the calcium ions is less than 1 wt. %, a shape-fixed effect becomes poor, affecting the shape recovery status. The concentration of the alginate 110 used in the first embodiment is, for example, 0.5 to 5 wt. %. If the concentration of the alginate is less than 0.5 wt. %, mechanical properties of the double network hydrogel are not significantly improved, and the effect of shape memory is not obvious. If the concentration of the alginate is greater than 5 wt. %, the excessively high concentration of the alginate affects the gelation effect of previous cellulose, interferes the reaction between the cellulose and the cross-linking agent, and reduces the stability of the hydrogel. In a preparation process of the highly compressible shape memory double network hydrogel 100, the cellulose 106 and the alginate 110 are mixed first, the chemical crosslinking is then performed to obtain the first network 102, followed by the physical crosslinking to obtain the second network 104. In the first embodiment, the highly compressible shape memory double network hydrogel 100 may also include a chelating agent. The chelating agent is, for example, ethylenediaminetetraacetic acid (EDTA), nitrilotriacetic acid, or hydroxyethylethylenediaminetriacetic acid (HEDTA). A preparation method of the highly compressible shape memory double network hydrogel 100 is described in detail below.

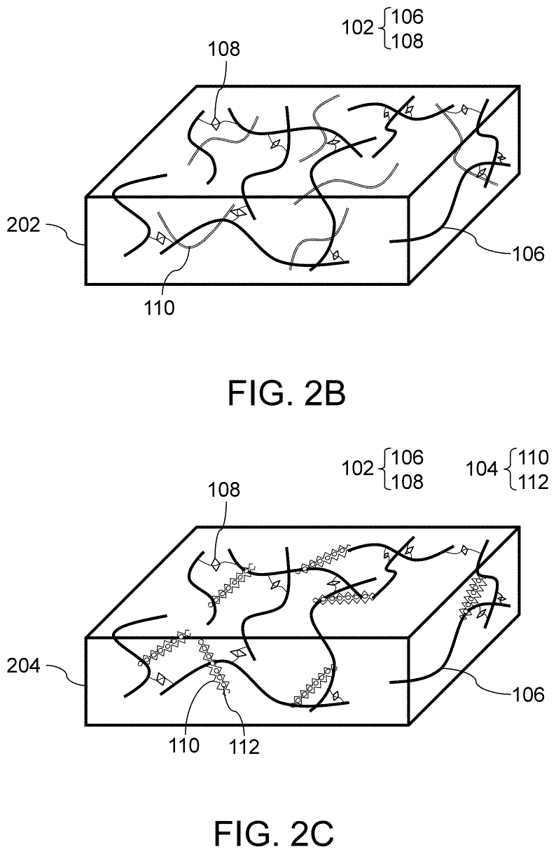

FIG. 2A to FIG. 2C are schematic diagrams of a preparation process of a highly compressible shape memory double network hydrogel according to a second embodiment of the disclosure, in which the reference numerals of the first embodiment are used to denote the same composition, and the description of the same composition can be referred to the relevant contents of the first embodiment and therefore will not be repeated in the following sections.

Referring to FIG. 2A first, the cellulose 106 and the alginate 110 are mixed to obtain a mixture 200, and the method of mixing the cellulose 106 and the alginate 110 includes adding alginate powder to a cellulose solution. Moreover, in order to allow the cellulose 106 and the subsequent addition of the cross-linking agent to react at room temperature, the cellulose solution may be repeatedly frozen and thawed for three to five times before mixing the cellulose 106 and the alginate 110.

Then, referring to FIG. 2B, the cellulose 106 in the mixture 200 is chemically crosslinked to form a hydrogel structure 202. Since the chemical crosslinking in the disclosure utilizes the epoxy group in the cross-linking agent, a cross-linking agent with epoxy groups, such as epichlorohydrin (ECH), poly(ethylene glycol) diglycidyl ether or diglycidyl ether, may be used. The concentration of the cross-linking agent is, for example, 5 to 10 wt. %.

Next, referring to FIG. 2C, the physical crosslinking is performed, so that the alginate 110 in the hydrogel structure 202 reacts with the divalent metal ions 112 to form a double network hydrogel 204. In addition, in terms of the effectiveness of the shape memory required, before proceeding with the step in FIG. 2C, the hydrogel structure 202 of FIG. 2B may be shaped, such as flattened or elongated, and subsequently, through the physical crosslinking with the divalent metal ions 112, the shape of the double network hydrogel 204 may be fixed to achieve the shape-fixed effect. In this embodiment, the divalent metal ions 112, if being calcium ions, may be sourced from one of calcium chloride, calcium sulfate, calcium aluminosilicate, calcium carbonate, calcium chloride, calcium oxide, calcium hydroxide, calcium lactate, calcium citrate, calcium gluconate, or a group thereof.

If high strength and high compressibility are required, a chelating agent such as ethylenediaminetetraacetic acid (EDTA) may be added to the double network hydrogel 204 after physical crosslinking instead of shaping before the step in FIG. 2C (the concentration of EDTA is, for example, 2 to 6 wt. %). By using the chelating agent with higher affinity for the divalent metal ions 112 (e.g. calcium ions), the divalent metal ions may be removed from the alginate structure for shaping the hydrogel structure. The chelating agent may also be changed to nitrilotriacetic acid or hydroxyethylethylenediaminetriacetic acid (HEDTA). Then, a solution containing the divalent metal ions is added again to the shaped double network hydrogel for fixing the shape in order to achieve the shape-fixed effect. However, the disclosure is not limited thereto. In another embodiment, the shaping step may be omitted, and the preparation of the double network hydrogel 204 may be completed directly.

Since the crosslinking sequence of the double network hydrogel 204 is to mix the cellulose 106 and the alginate 110 first, and to perform the physical crosslinking after the chemical crosslinking, the resulting product has high compressibility and also has shape memory effect. The following mechanical performance tests are used to verify effectiveness.

Ingredient:

| | Molecular weight, Mw | | |
|---|---|---|---|
| Chemicals | (g/mol) | Manufacturer | model |
| Cellulose | 203.2 | Sigma (SIG) | AL-435236 |
| (microcrystalline) | | | |
| Epichlorohydrin | 92.52 | Alfa Aesar | AF-A15823 |
| (ECH) | | | |
| Sodium alginate | 216.12 | Alfa Aesar | AF-A18565 |
| Sodium Hydroxide | 39.997 | ECHO | 1943-0150 |
| (NaOH) | | | |
| Calcium chloride | 110.98 | Sigma-Aldrich | SI-C4901 |
| (CaCl₂) | | | |
| Urea | 60.06 | Alfa Acsar | AF-36428 |

The ingredients are stored at room temperature.

### Experimental Example 1

First, 0.8 g of NaOH and 0.4 g of urea were added to 10 mL of deionized water to prepare a solution of 4 wt. % urea/8 wt. % NaOH.

0.4 g (the concentration of 4 wt. %) of cellulose was added to the solution and mixed for 15 minutes to obtain a cellulose solution.

The cellulose solution is stored at −80° C. for 24 hours, and then the cellulose solution is repeatedly frozen and thawed for three to five times. This step is to allow the cellulose and a cross-linking agent to react at room temperature. If the number of freezing/thawing is less than three times, the reaction between the cellulose and the cross-linking agent needs to be performed at 60° C. in order to form the gel. However, if the number of freezing/thawing is greater than five, the reactivity of the hydroxyl group on the cellulose side chain is reduced, making the cellulose solution unable to be gelled.

Next, 0.2 g (the concentration of 2 wt. %) of alginate powder was added to 10 mL of the cellulose solution, and stirred for 30 minutes to obtain a mixture.

0.8 mL of epichlorohydrin (ECH)(the concentration of 8 wt. %) was added to the mixture and stirred for 15 minutes at room temperature to become a hydrogel, and then the hydrogel was stored in a specific mold.

Then, the hydrogel was immersed in a 4 wt. % calcium chloride solution (calcium ions concentration of 4 wt. %) for two hours to obtain a double network hydrogel.

### Comparative Example 1

The steps of Experimental Example 1 were followed, but no alginate powder was added, nor was the hydrogel immersed in calcium phosphate. Therefore, what was obtained was a single network hydrogel after chemical crosslinking of the cellulose.

### Comparative Example 2

The ingredients of Experimental Example 1 were used, but the preparation process was as follows.

First, 0.8 g of NaOH and 0.4 g of urea were added to 10 mL of deionized water to prepare a solution of 4 wt. % urea/8 wt. % NaOH.

0.4 g (the concentration of 4 wt. %) of cellulose was added to the solution and mixed for 15 minutes to obtain a cellulose solution.

The cellulose solution is stored at −80° C. for 24 hours, and then the cellulose solution is repeatedly frozen and thawed for three to five times. This step is to allow the cellulose and the cross-linking agent to react at room temperature. If the number of freezing/thawing is less than three times, the reaction between the cellulose and the cross-linking agent needs to be performed at 60 degrees in order to form the gel. However, if the number of freeze/thaw is greater than five, the reactivity of the hydroxyl group on the cellulose side chain is reduced, making the cellulose solution unable to be gelled.

Next, 0.8 mL of ECH was added to the cellulose solution and stirred for 15 minutes at room temperature, and then stored in a specific mold for 24 hours to become a cellulose hydrogel.

0.4 g (the concentration of 4 wt. %) of alginate powder was added to 10 mL of the deionized water to form an alginate solution.

Then, the cellulose hydrogel was immersed in the alginate solution and stirred for about 24 hours.

After that, the hydrogel was immersed in a calcium chloride solution with a concentration of 4 wt. % for two hours to obtain a double network hydrogel.

<Mechanical Strength>

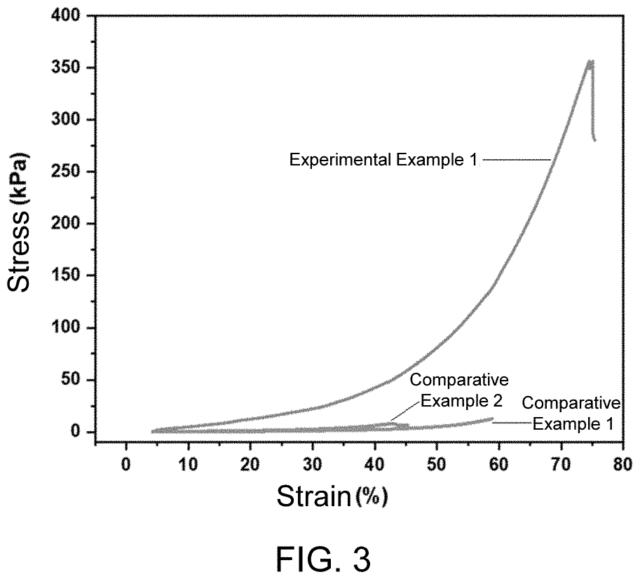

Samples of Experimental Example 1, Comparative Example 1, and Comparative Example 2 were subjected to stress-strain curves, and the results are shown in FIG. 3.

It can be seen from FIG. 3 that the single network hydrogel of Comparative Example 1 is much weaker than the double network hydrogel of Experimental Example 1 in terms of strength and ductility. Moreover, even if both are double network hydrogel, the hydrogel strength of Comparative Example 2 is obviously lower than the hydrogel strength of Experimental Example 1, representing the different addition orders of crosslinking agent and alginate solution also largely affect mechanical properties.

<Cyclic Compression Test>

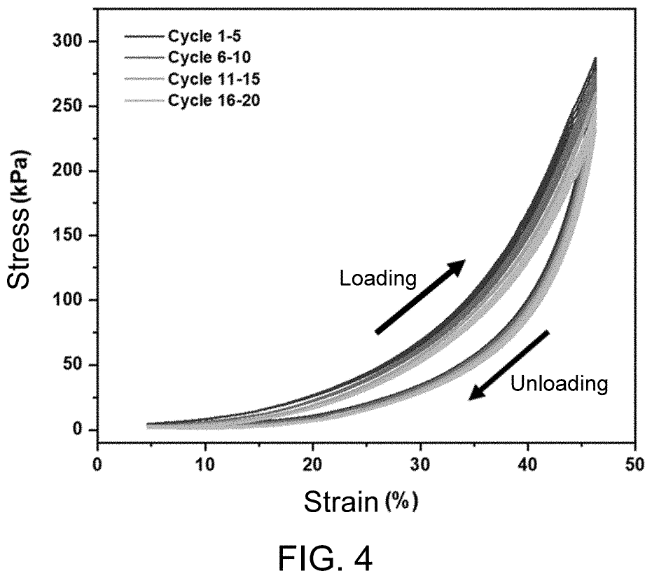

The sample of Experimental Example 1 was subjected to a cyclic compression test, and the results are shown in FIG. 4, where an area between loading and unloading curves represents energy consumption per unit volume. “Cycle 1-5” in FIG. 4 represents a stress-strain curve from a first cyclic compression to a fifth cyclic compression of the hydrogel, so there are five curves stacked together; and so on.

It can be seen from FIG. 4 that the double network hydrogel of Experimental Example 1 can still recover to its original shape after 50% strain. Although the double network hydrogel of Experimental Example 1 shows hysteresis loops after 20 cycles of loading and unloading, the integrity of the hydrogel remains intact and return to its original shape without permanent deformation.

With the above characteristics verified by experiments, the highly compressible shape memory double network hydrogel of the disclosure may be applied to human tissues or organs that need to withstand strong compressive stress or load, such as intervertebral discs (cervical vertebrae, lumbar vertebrae, etc.) and articular cartilage.

When highly compressed, the double network hydrogel has good resilience and is therefore particularly suitable for use as a tissue graft that requires repetitive compression, such as an intervertebral disk scaffold. Thus, an intervertebral disk scaffold according to a third embodiment of the disclosure includes the highly compressible shape memory double network hydrogel. In addition, the highly compressible shape memory double network hydrogel of the disclosure also has shape memory properties that facilitate the surgical implantation process and reduce the difficulty, thus simplifying the complicated surgical procedures and reducing risks, pain and complications.

For example, in order to facilitate implantation into a small space (e.g. the space between cervical vertebrae), the highly compressible shape memory double network hydrogel of the disclosure may be shaped into a small sheet, which is then used by first placing it into an accommodation space (e.g. between cervical vertebrae and lumbar vertebrae) and then adding a chelating agent to the highly compressible shape memory double network hydrogel to expand the sheet back to its original shape, in which the chelating agent is, for example, EDTA. In another embodiment, the highly compressible shape memory double network hydrogel of the disclosure may also be placed directly into the mold without being shaped, and no chelating agent is added to retain better strength and compressibility.

The following biological experiments were conducted to demonstrate the efficacy of the highly compressible shape memory double network hydrogel for the intervertebral disk scaffold.

<Magnetic Resonance Imaging (MRI) Test>

The following table shows the details of each group of MRI test subjects.

| Group | Information |
|---|---|
| Discectomy control | Intervertebral discs of a rat were removed and |
| group (DC) | injected with PBS. |
| Double network | The intervertebral discs were replaced with |
| hydrogel | the double network |
| group (DN) | hydrogel of Experimental Example 1. |
| Peptidated | The intervertebral discs were replaced |
| double | with a double network hydrogel |
| network | containing functional peptides, |
| hydrogel | in which the double network hydrogel |
| group | was the double network hydrogel |
| (PDN) | used in Experimental Example 1. |
| GDF-5 combined | The intervertebral discs were replaced |
| with | with a double network |
| peptide- | hydrogel containing functional peptides |
| functionalized | and bioactive molecules, |
| double network | in which the double network hydrogel |
| hydrogel | was the double network |
| group (GPDN) | hydrogel used in Experimental Example 1. |

In the table, DC represents a control group in which the intervertebral discs were removed, and no material was implanted; DN represents an experimental group in which the intervertebral discs were replaced with the double network hydrogel of Experimental Example 1; PDN and GPDN represent experimental groups in which the intervertebral discs were replaced with hydrogels carrying growth factors or different therapeutic factors, respectively.

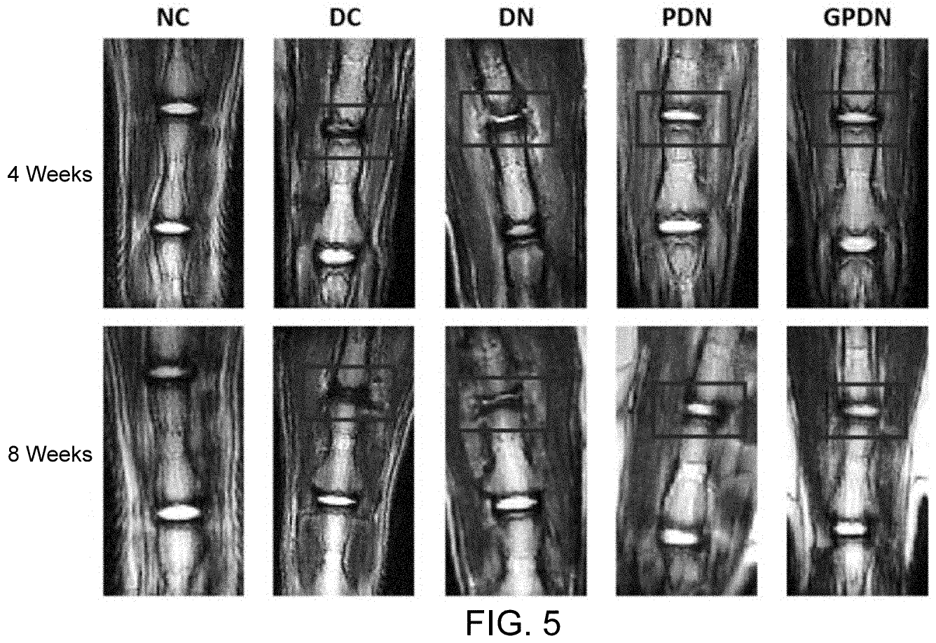

Then, a period of time after the double network hydrogel was implanted in the caudal vertebrae of the rats, T2-MRI was used to directly observe the morphology and water content of the implanted device. The results were shown in FIG. 5, in which the stronger white signal indicates a higher water content. The boxed area is the location of the double network hydrogel implantation, and NC represents an image of the caudal vertebrae of a healthy rat.

As can be seen from FIG. 5, after eight weeks of implantation, the hydrogel remained at the implantation site and maintained a high-water content as observed by MRI. With the help of the growth factors or different therapeutic factors, it can also be found that the hydrogel can effectively support damaged part and maintain the integrity of the hydrogel.

<Immunostaining (IHC) Test>

Immunostaining was used to observe whether new intervertebral disc tissue was generated in the caudal vertebrae of the rat. Target proteins are Vimentin (green) and FOXF1 (Forkhead Box protein F1, red), both of which are specific proteins commonly found in healthy intervertebral discs, so this method may be used to determine whether the implanted double network hydrogel has a therapeutic effect on intervertebral disc regeneration and repair.

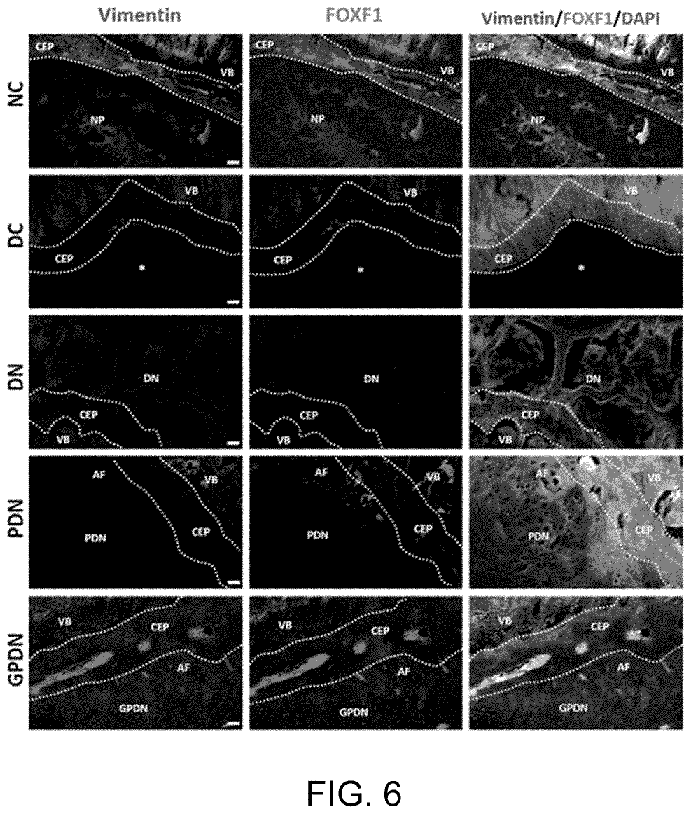

FIG. 6 shows stained images of five experiments (NC, DC, DN, PDN, and GPDN) after eight weeks, where VB refers to vertebral bodies, NP refers to nucleus pulposus, CEP refers to cartilage endplate, and AF refers to annulus fibrosus. From the staining results in FIG. 6, it can be found that in the hydrogel groups added with the growth factors or different therapeutic factors (PDN, GPDN), there is a significant increase in area of red and green.

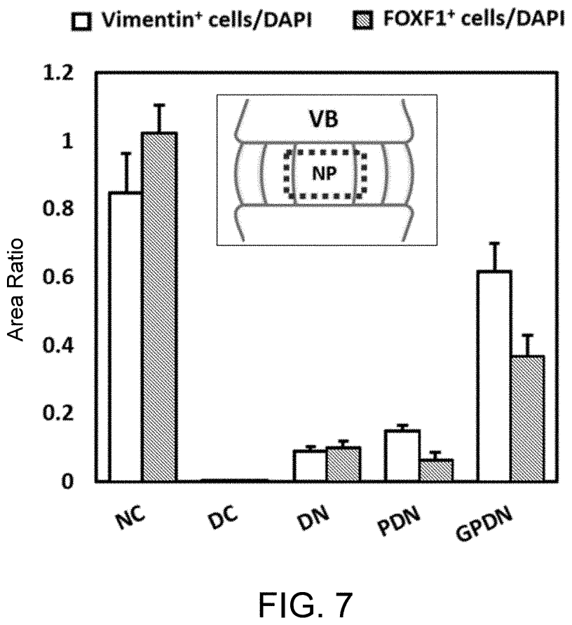

For more effective analysis, the red and green areas in FIG. 6 were quantified by image analysis software (ImageJ) to obtain FIG. 7.

As can be seen from FIG. 7, DN, PDN and GPDN all contain Vimentin and FOXF1 proteins, and the results in the group with added growth factors or different therapeutic factors (e.g., GPDN) also found the content of Vimentin and FOXF1 proteins close to that of normal healthy intervertebral discs. Therefore, it has been proved that the highly compressible shape memory double network hydrogel of the disclosure may not only support the damaged part of the intervertebral disc, but also grow new intervertebral disc tissue with the help of growth factors or different therapeutic factors at the same time, thus becoming a choice of intervertebral disc replacement.

To sum up, the disclosure uses natural polymers as ingredients, and through a specific crosslinking sequence, the prepared double network hydrogel has high compressibility and shape memory function. The double network hydrogel of the disclosure is mainly composed of chemical crosslinking and physical crosslinking. Chemical crosslinking forms a hard segment to stabilize the hydrogel, which is responsible for controlling the permanent shape, while physical crosslinking forms a soft segment, which is reversible and determines the temporary shape of the hydrogel. Therefore, the double network hydrogel of the disclosure can be applied zto intervertebral disk scaffolds that are subject to higher compressive stress.

It will be apparent to those skilled in the art that various modifications and variations can be made to the disclosed without departing from the scope or spirit of the disclosure. In view of the foregoing, it is intended that the disclosure covers modifications and variations provided that they fall within the scope of the following claims and their equivalents.

What is claimed is:

1. A highly compressible shape memory double network hydrogel, comprising a first network and a second network interpenetrating with each other, wherein
the first network is a chemically crosslinked cellulose obtained by chemical crosslinking, and the chemical crosslinking is accomplished by formation of ether groups in the cellulose; and the second network is a physically crosslinked alginate obtained by physical crosslinking, and the physical crosslinking is accomplished by reaction of the alginate with divalent metal ions,
in a preparation process of the highly compressible shape memory double network hydrogel, the cellulose and the alginate are mixed first, the chemical crosslinking is then performed to obtain the first network, followed by the physical crosslinking to obtain the second network.

2. The highly compressible shape memory double network hydrogel according to claim 1, wherein a concentration of the cellulose is 1 to 10 wt. %.

3. The highly compressible shape memory double network hydrogel according to claim 1, further comprising a cross-linking agent for the chemical crosslinking, wherein a concentration of the cross-linking agent is 5 to 10 wt. %.

4. The highly compressible shape memory double network hydrogel according to claim 3, wherein the cross-linking agent comprises epichlorohydrin (ECH), poly(ethylene glycol) diglycidyl ether or diglycidyl ether.

5. The highly compressible shape memory double network hydrogel according to claim 1, wherein the divalent metal ions are calcium ions, copper ions, ferrous ions, manganese ions, magnesium ions, strontium ions or zinc ions.

6. The highly compressible shape memory double network hydrogel according to claim 5, wherein a concentration of the calcium ions (Ca²⁺) is 1 to 10 wt. %, and a concentration of the alginate is 0.5 to 5 wt. %.

7. The highly compressible shape memory double network hydrogel according to claim 1 further comprising a chelating agent.

8. The highly compressible shape memory double network hydrogel according to claim 7, wherein the chelating agent comprises ethylenediaminetetraacetic acid (EDTA), nitrilotriacetic acid, or hydroxyethylethylenediaminetriacetic acid (HEDTA).

9. An intervertebral disk scaffold comprising the highly compressible shape memory double network hydrogel according to claim 1.

10. A method of using the highly compressible shape memory double network hydrogel according to claim 1, comprising:
placing the highly compressible shape memory double network hydrogel into a mold; and
adding a chelating agent to the highly compressible shape memory double network hydrogel.

11. The method according to claim 10, wherein the chelating agent comprises ethylenediaminetetraacetic acid (EDTA), nitrilotriacetic acid, or hydroxyethylethylenediaminetriacetic acid (HEDTA).

12. A preparation method of a highly compressible shape memory double network hydrogel, comprising:
mixing cellulose and an alginate to obtain a mixture;
performing a chemical crosslinking on the cellulose in the mixture to form a hydrogel structure; and
performing a physical crosslinking after the chemical crosslinking to react the alginate in the hydrogel structure with divalent metal ions for forming a double network structure.

13. The preparation method according to claim 12, wherein a cross-linking agent used in the chemical crosslinking comprises epichlorohydrin (ECH), poly(ethylene glycol) diglycidyl ether or diglycidyl ether.

14. The preparation method according to claim 12, wherein the divalent metal ions are calcium ions, copper ions, ferrous ions, manganese ions, magnesium ions, strontium ions or zinc ions.

15. The preparation method according to claim 12, wherein before performing the physical crosslinking, further comprises shaping the hydrogel structure.

16. The preparation method according to claim 12, wherein after performing the physical crosslinking further comprises:
adding a chelating agent in the double network hydrogel;
shaping the hydrogel structure; and
adding a solution containing the divalent metal ions to the shaped double network hydrogel.

17. The preparation method according to claim 16, wherein the chelating agent comprises ethylenediaminetetraacetic acid (EDTA), nitrilotriacetic acid, or hydroxyethylethylenediaminetriacetic acid (HEDTA).

18. The preparation method according to claim 12, wherein a method of mixing the cellulose and the alginate comprises adding alginate powder to a cellulose solution.

19. The preparation method according to claim 18, wherein before mixing the cellulose and the alginate, further comprises repeatedly freezing and thawing the cellulose solution three to five times.
